(19) United States
(12) Patent Application Publication
(10) Pub. No.: US 2025/0183712 A1
(43) Pub. Date: Jun. 5, 2025

(54) IMPLANT WITH MAGNETIC INDUCTION ANTENNA

(71) Applicant: Cochlear Limited, Macquarie University, NSW (AU)

(72) Inventors: Charles Roger Aaron Leigh, North Epping (AU); Werner Meskens, Opwijk (BE)

(21) Appl. No.: 18/838,819

(22) PCT Filed: Feb. 22, 2023
(86) PCT No.: PCT/IB2023/051585

§ 371 Date: Aug. 15, 2024

Related U.S. Application Data
(60) Provisional application No. 63/269,230, filed on Mar. 11, 2022.

(51) Int. Cl. H02J 50/10; A61N 1/36; A61N 1/372; A61N 1/378; H01F 27/28; H01Q 7/06; H02J 50/40; H02J 50/80

(52) U.S. Cl. CPC H02J 50/10; A61N 1/36038; A61N 1/37229; A61N 1/3787; H01F 27/2823; H01Q 7/06; H02J 50/402; H02J 50/80

(57) ABSTRACT

An apparatus includes at least one implantable housing containing circuitry and configured to be implanted on and substantially parallel to a bone surface within a recipient. The apparatus further includes at least one electrical conduit in electrical communication with the circuitry and extending from the at least one implantable housing to a region within the recipient. The apparatus further includes at least one magnetic induction antenna in electrical communication with the at least one electrical conduit and spaced from the at least one implantable housing. The at least one magnetic induction antenna extends around an antenna axis. The at least one magnetic induction antenna is configured to be affixed within the region with the antenna axis substantially non-parallel and substantially non-orthogonal to the bone surface.

### BACKGROUND

### Field

The present application relates generally to systems and methods for wirelessly communicating data to and/or from a device implanted on or within a recipient's body.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises at least one implantable housing containing circuitry and configured to be implanted on and substantially parallel to a bone surface within a recipient. The apparatus further comprises at least one electrical conduit in electrical communication with the circuitry and extending from the at least one implantable housing to a region within the recipient. The apparatus further comprises at least one magnetic induction antenna in electrical communication with the at least one electrical conduit and spaced from the at least one implantable housing. The at least one magnetic induction antenna extends around an antenna axis. The at least one magnetic induction antenna is configured to be affixed within the region with the antenna axis substantially non-parallel and substantially non-orthogonal to the bone surface.

In another aspect disclosed herein, a method comprises receiving, using a plurality of implanted magnetic induction antennas, corresponding portions of a magnetic flux from at least one external magnetic induction antenna. The method further comprises detecting the amplitude of an electric signal induced within each of the implanted magnetic induction antennas by the corresponding received portion of the magnetic flux. The method further comprises determining which of the implanted magnetic induction antennas has the largest induced signal amplitude. The method further comprises selecting the implanted magnetic induction antenna corresponding to the largest induced signal amplitude.

In another aspect disclosed herein, an apparatus comprises at least one external device configured to be worn by a recipient. The at least one external device comprises at least one external magnetic induction antenna configured to generate a magnetic flux. The apparatus further comprises at least one implantable device comprising circuitry and at least a first magnetic induction antenna and a second magnetic induction antenna in electrical communication with the circuitry. The first and second magnetic induction antennas are affixed to one another. The first magnetic induction antenna has a first antenna axis and the second magnetic induction antenna has a second antenna axis substantially non-parallel to the first antenna axis.

### BRIEF DESCRIPTION OF THE DRAWINGS

Implementations are described herein in conjunction with the accompanying drawings, in which:

FIG. 1A is a perspective view of an example cochlear implant auditory prosthesis implanted in a recipient in accordance with certain implementations described herein;

FIG. 1B is a perspective view of an example fully implantable middle ear implant auditory prosthesis implanted in a recipient in accordance with certain implementations described herein;

FIGS. 2A and 2B schematically illustrate a cross-sectional view and a top view, respectively, of an example apparatus in accordance with certain implementations described herein;

FIGS. 3A-3I schematically illustrate various examples of the at least one MI antenna in accordance with certain implementations described herein; and

FIG. 4 is a flow diagram of an example method in accordance with certain implementations described herein.

### DETAILED DESCRIPTION

Certain implementations described herein provide a medical implant having at least one magnetic induction (MI) antenna configured to be implanted within a recipient's body and configured to be in communication with an external MI antenna while avoiding being in a dead zone of the external MI antenna. The at least one implanted MI antenna can be on a malleable lead extending from an implantable housing (e.g., positioned on a skull bone surface) such that the at least one implantable MI antenna can be positioned in a region (e.g., mastoid cavity) spaced from the implantable housing with a position and/or orientation configured to facilitate communication with the external MI antenna. The at least one implanted MI antenna can comprise a plurality of antenna coils that are oriented at least 45 degrees from one another (e.g., two antenna coils in a “V” or “T” formation) and the implant can be configured to select among the antenna coils to utilize an antenna coil having a sufficiently large coupling with the external MI antenna.

The teachings detailed herein are applicable, in at least some implementations, to any type of implantable or non-implantable stimulation system or device (e.g., implantable or non-implantable auditory prosthesis device or system). Implementations can include any type of medical device that can utilize the teachings detailed herein and/or variations thereof. Furthermore, while certain implementations are described herein in the context of auditory prosthesis devices, certain other implementations are compatible in the context of other types of devices or systems.

Merely for ease of description, apparatus and methods disclosed herein are primarily described with reference to an illustrative medical device, namely an implantable transducer assembly including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Implementations can include any type of auditory prosthesis that can utilize the teachings detailed herein and/or variations thereof. Certain such implementations can be referred to as “partially implantable,” “semi-implantable,” “mostly implantable,” “fully implantable,” or “totally implantable” auditory prostheses. In some implementations, the teachings detailed herein and/or variations thereof can be utilized in other types of prostheses beyond auditory prostheses.

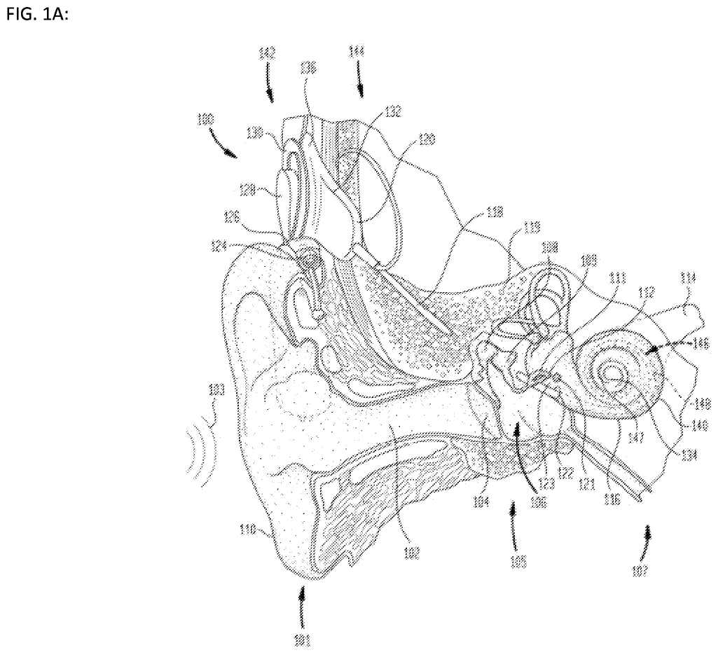

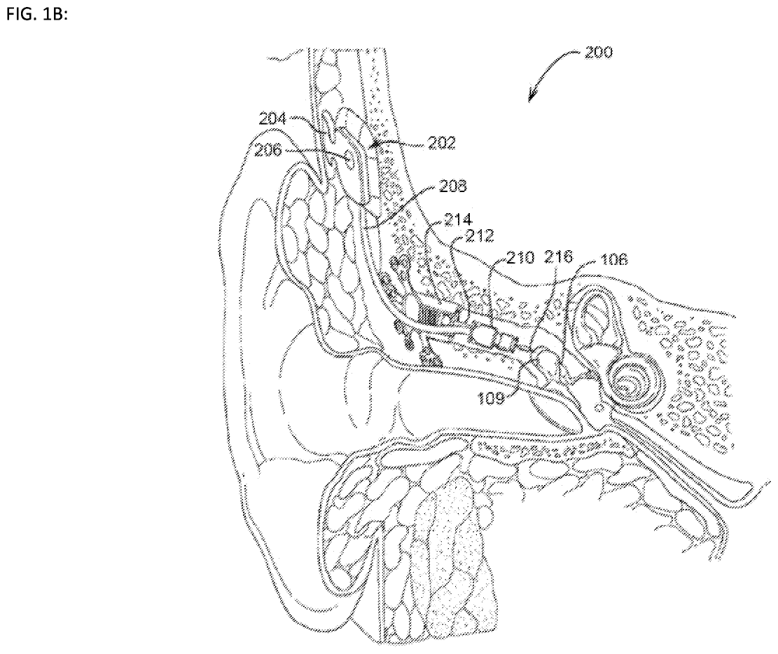

FIG. 1A is a perspective view of an example cochlear implant auditory prosthesis 100 implanted in a recipient in accordance with certain implementations described herein. The example auditory prosthesis 100 is shown in FIG. 1A as comprising an implanted stimulator unit 120 and a microphone assembly 124 that is external to the recipient (e.g., a partially implantable cochlear implant). An example auditory prosthesis 100 (e.g., a totally implantable cochlear implant; a mostly implantable cochlear implant) in accordance with certain implementations described herein can replace the external microphone assembly 124 shown in FIG. 1A with a subcutaneously implantable microphone assembly, as described more fully herein. In certain implementations, the example cochlear implant auditory prosthesis 100 of FIG. 1A can be in conjunction with a reservoir of liquid medicament as described herein.

As shown in FIG. 1A, the recipient has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1A, the example auditory prosthesis 100 comprises one or more components which are temporarily or permanently implanted in the recipient. The example auditory prosthesis 100 is shown in FIG. 1A with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more sound input elements (e.g., an external microphone 124) for detecting sound, a sound processing unit 126 (e.g., disposed in a Behind-The-Ear unit), a power source (not shown), and an external transmitter unit 128. In the illustrative implementations of FIG. 1A, the external transmitter unit 128 comprises an external coil 130 (e.g., a wire antenna coil comprising multiple turns of electrically insulated single-strand or multi-strand platinum or gold wire) and, preferably, a magnet (not shown) secured directly or indirectly to the external coil 130. The external coil 130 of the external transmitter unit 128 is part of an inductive radio frequency (RF) communication link with the internal component 144. The sound processing unit 126 processes the output of the microphone 124 that is positioned externally to the recipient's body, in the depicted implementation, by the recipient's auricle 110. The sound processing unit 126 processes the output of the microphone 124 and generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate electrode assembly 118. In some implementations, the internal receiver unit 132 and the stimulator unit 120 are hermetically sealed within a biocompatible housing. The internal receiver unit 132 comprises an internal coil 136 (e.g., a wire antenna coil comprising multiple turns of electrically insulated single-strand or multi-strand platinum or gold wire), and preferably, a magnet (also not shown) fixed relative to the internal coil 136. The internal receiver unit 132 and the stimulator unit 120 are hermetically sealed within a biocompatible housing, sometimes collectively referred to as a stimulator/receiver unit. The internal coil 136 receives power and/or data signals from the external coil 130 via a transcutaneous energy transfer link (e.g., an inductive RF link). The stimulator unit 120 generates electrical stimulation signals based on the data signals, and the stimulation signals are delivered to the recipient via the elongate electrode assembly 118.

The elongate electrode assembly 118 has a proximal end connected to the stimulator unit 120, and a distal end implanted in the cochlea 140. The electrode assembly 118 extends from the stimulator unit 120 to the cochlea 140 through the mastoid bone 119. In some implementations, the electrode assembly 118 may be implanted at least in the basal region 116, and sometimes further. For example, the electrode assembly 118 may extend towards apical end of cochlea 140, referred to as cochlea apex 134. In certain circumstances, the electrode assembly 118 may be inserted into the cochlea 140 via a cochleostomy 122. In other circumstances, a cochleostomy may be formed through the round window 121, the oval window 112, the promontory 123, or through an apical turn 147 of the cochlea 140.

The elongate electrode assembly 118 comprises a longitudinally aligned and distally extending array 146 of electrodes or contacts 148, sometimes referred to as electrode or contact array 146 herein, disposed along a length thereof. Although the electrode array 146 can be disposed on the electrode assembly 118, in most practical applications, the electrode array 146 is integrated into the electrode assembly 118 (e.g., the electrode array 146 is disposed in the electrode assembly 118). As noted, the stimulator unit 120 generates stimulation signals which are applied by the electrodes 148 to the cochlea 140, thereby stimulating the auditory nerve 114.

While FIG. 1A schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

FIG. 1B schematically illustrates a perspective view of an example fully implantable auditory prosthesis 200 (e.g., fully implantable middle ear implant or totally implantable acoustic system), implanted in a recipient, utilizing an acoustic actuator in accordance with certain implementations described herein. The example auditory prosthesis 200 of FIG. 1B comprises a biocompatible implantable assembly 202 (e.g., comprising an implantable capsule) located subcutaneously (e.g., beneath the recipient's skin and on a recipient's skull). While FIG. 1B schematically illustrates an example implantable assembly 202 comprising a microphone, in other example auditory prostheses 200, a pendant microphone can be used (e.g., connected to the implantable assembly 202 by a cable). The implantable assembly 202 includes a signal receiver 204 (e.g., comprising a coil element) and an acoustic transducer 206 (e.g., a microphone comprising a diaphragm and an electret or piezoelectric transducer) that is positioned to receive acoustic signals through the recipient's overlying tissue. The implantable assembly 202 may further be utilized to house a number of components of the fully implantable auditory prosthesis 200. For example, the implantable assembly 202 can include an energy storage device and a signal processor (e.g., a sound processing unit). Various additional processing logic and/or circuitry components can also be included in the implantable assembly 202 as a matter of design choice.

For the example auditory prosthesis 200 shown in FIG. 1B, the signal processor of the implantable assembly 202 is in operative communication (e.g., electrically interconnected via a wire 208) with an actuator 210 (e.g., comprising a transducer configured to generate mechanical vibrations in response to electrical signals from the signal processor). In certain implementations, the example auditory prosthesis 100, 200 shown in FIGS. 1A and 1B can comprise an implantable microphone assembly, such as the microphone assembly 206 shown in FIG. 1B. For such an example auditory prosthesis 100, the signal processor of the implantable assembly 202 can be in operative communication (e.g., electrically interconnected via a wire) with the microphone assembly 206 and the stimulator unit of the main implantable component 120. In certain implementations, at least one of the microphone assembly 206 and the signal processor (e.g., a sound processing unit) is implanted on or within the recipient.

The actuator 210 of the example auditory prosthesis 200 shown in FIG. 1B is supportably connected to a positioning system 212, which in turn, is connected to a bone anchor 214 mounted within the recipient's mastoid process (e.g., via a hole drilled through the skull). The actuator 210 includes a connection apparatus 216 for connecting the actuator 210 to the ossicles 106 of the recipient. In a connected state, the connection apparatus 216 provides a communication path for acoustic stimulation of the ossicles 106 (e.g., through transmission of vibrations from the actuator 210 to the incus 109).

During normal operation, ambient acoustic signals (e.g., ambient sound) impinge on the recipient's tissue and are received transcutaneously at the microphone assembly 206. Upon receipt of the transcutaneous signals, a signal processor within the implantable assembly 202 processes the signals to provide a processed audio drive signal via wire 208 to the actuator 210. As will be appreciated, the signal processor may utilize digital processing techniques to provide frequency shaping, amplification, compression, and other signal conditioning, including conditioning based on recipient-specific fitting parameters. The audio drive signal causes the actuator 210 to transmit vibrations at acoustic frequencies to the connection apparatus 216 to affect the desired sound sensation via mechanical stimulation of the incus 109 of the recipient.

The subcutaneously implantable microphone assembly 202 is configured to respond to auditory signals (e.g., sound; pressure variations in an audible frequency range) by generating output signals (e.g., electrical signals; optical signals; electromagnetic signals) indicative of the auditory signals received by the microphone assembly 202, and these output signals are used by the auditory prosthesis 100, 200 to generate stimulation signals which are provided to the recipient's auditory system. To compensate for the decreased acoustic signal strength reaching the microphone assembly 202 by virtue of being implanted, the diaphragm of an implantable microphone assembly 202 can be configured to provide higher sensitivity than are external non-implantable microphone assemblies. For example, the diaphragm of an implantable microphone assembly 202 can be configured to be more robust and/or larger than diaphragms for external non-implantable microphone assemblies.

The example auditory prostheses 100 shown in FIG. 1A utilizes an external microphone 124 and the auditory prosthesis 200 shown in FIG. 1B utilizes an implantable microphone assembly 206 comprising a subcutaneously implantable acoustic transducer. In certain implementations described herein, the auditory prosthesis 100 utilizes one or more implanted microphone assemblies on or within the recipient. In certain implementations described herein, the auditory prosthesis 200 utilizes one or more microphone assemblies that are positioned external to the recipient and/or that are implanted on or within the recipient, and utilizes one or more acoustic transducers (e.g., actuator 210) that are implanted on or within the recipient. In certain implementations, an external microphone assembly can be used to supplement an implantable microphone assembly of the auditory prosthesis 100, 200. Thus, the teachings detailed herein and/or variations thereof can be utilized with any type of external or implantable microphone arrangement, and the acoustic transducers shown in FIGS. 1A and 1B are merely illustrative.

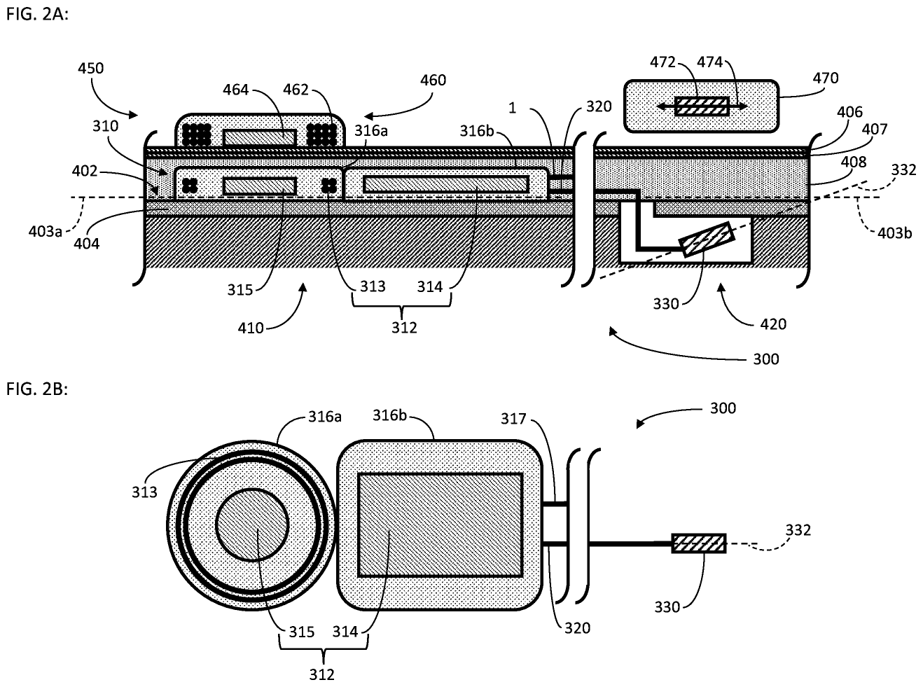

FIGS. 2A and 2B schematically illustrate a cross-sectional view and a top view, respectively, of an example apparatus 300 in accordance with certain implementations described herein. The apparatus 300 comprises at least one implantable housing 310 containing circuitry 312 and configured to be implanted on and substantially parallel to a bone surface 402 (e.g., a surface of the skull 404) within a recipient. The apparatus 300 further comprises at least one electrical conduit 320 in electrical communication with the circuitry 312 and extending from the at least one implantable housing 310 to a region 420 within the recipient. The apparatus 300 further comprises at least one magnetic induction (MI) antenna 330 in electrical communication with the at least one electrical conduit 320 and spaced from the at least one implantable housing 310. The at least one MI antenna 330 extends around an antenna axis 332, the at least one MI antenna 330 configured to be affixed within the region 420 with the antenna axis 332 substantially non-parallel and substantially non-orthogonal to the bone surface 402.

The example apparatus 300 is shown in FIG. 2A as part of a transcutaneous system (e.g., auditory prosthesis system) comprising the example apparatus 300 (e.g., an implanted portion of an acoustic prosthesis system) and at least one external device 450 (e.g., an external portion of the acoustic prosthesis system). The apparatus 300 can comprise one or more active elements (e.g., stimulator unit 120; assembly 202; vibrating actuator; not shown in FIG. 2A) configured to deliver stimuli to the recipient's body and/or to detect an attribute or condition of the recipient's body.

In certain implementations, the at least one external device 450 can comprise a first portion 460 configured to be worn on the recipient's skin 406 over the implantable housing 310 and a second portion 470 configured to be worn in proximity to (e.g., over) the at least one MI antenna 330, the at least one external device 450 configured to provide power and/or data to the apparatus 300 and/or to receive data from the apparatus 300. In certain implementations, the first portion 460 and the second portion 470 are separate from one another (e.g., as shown in FIG. 2A), while in certain other implementations, the first portion 460 and the second portion 470 are within a common housing or within two housings that are attached to one another (e.g., electrically connected by at least one electrical conductor).

The first portion 460 (e.g., an “on-the-go” or OTG sound processor comprising driver circuitry) can comprise an energy transmission coil 462 and an external magnetic material 464. For example, the first portion 460 can comprise a biocompatible (e.g., skin-friendly) housing configured to hermetically seal the energy transmission coil 462 and/or the external magnetic material 464 from an environment surrounding the housing. The housing can also be substantially transparent to the electromagnetic or magnetic fields generated by the energy transmission coil 462 such that the housing does not substantially interfere with the power transmission to the apparatus 300.

The energy transmission coil 462 can comprise multiple turns of electrically insulated single-strand or multi-strand copper wire (e.g., a planar electrically conductive wire with multiple windings having a substantially circular, rectangular, spiral, or oval shape or other shape) or copper traces on epoxy of a printed circuit board. For example, the energy transmission coil 462 can have a diameter, length, and/or width (e.g., along a lateral direction substantially parallel to the recipient's skin 406) less than or equal to 60 millimeters (e.g., in a range of 15 millimeters to 40 millimeters; in a range of 25 millimeters to 50 millimeters; in a range of less than 30 millimeters; in a range of 20 millimeters to 60 millimeters; in a range greater than 60 millimeters).

The at least one magnetic material 464 of the first portion 460 can be configured to interact with a portion of the apparatus 300 (e.g., at least one internal magnetic material 315) when the first portion 460 is positioned on or over the skin 406 of the recipient above the apparatus 300 to generate a magnetic restoring force which holds the first portion 460 on the recipient's skin 406 such that the energy transmission coil 462 is in operative wireless communication with the apparatus 300 to wirelessly and transcutaneously transfer energy from the first portion 460 to the apparatus 300 (e.g., via a radio-frequency or RF link).

The second portion 470 (e.g., a “behind-the-ear” or BTE sound processor comprising driver circuitry and is held in place on and/or behind the recipient's pinna by a hook portion of the second portion 470) can comprise at least one external magnetic induction (MI) antenna 472 configured to interact with the at least one MI antenna 330 of the apparatus 300 to wirelessly and transcutaneously transfer data from the second portion 470 to the apparatus 300 and/or from the apparatus 300 to the second portion 470.

For example, the first portion 460 and/or the second portion 470 can comprise circuitry comprising one or more microprocessors (e.g., application-specific integrated circuits; generalized integrated circuits programmed by software with computer executable instructions; microelectronic circuitry; microcontrollers) and at least one storage device (e.g., at least one tangible or non-transitory computer readable storage medium; read only memory; random access memory; flash memory) configured to store information (e.g., data; commands) accessed by the one or more microprocessors during operation. The at least one storage device can be encoded with software (e.g., a computer program downloaded as an application) comprising computer executable instructions for instructing the one or more microprocessors (e.g., executable data access logic, evaluation logic, and/or information outputting logic). In certain implementations, the one or more microprocessors execute the instructions of the software to provide functionality as described herein. The circuitry can be configured to receive status data signals wirelessly communicated from the apparatus 300 via the at least one MI antenna 330 and the at least one external MI antenna 472. Furthermore, the circuitry can be configured to, in response to user input and/or conditions during operation, generate and wirelessly transmit control signals to the apparatus 300 via the at least one external MI antenna 472 and the at least one MI antenna 330 (e.g., to set or adjust operational parameters of the apparatus 300).

In certain implementations, the at least one implantable housing 310 is configured to be positioned beneath tissue of the recipient's body. For example, as shown in FIG. 2A, the at least one implantable housing 310 is beneath the skin 406, fat 407, and/or muscular 408 layers and above and on a bone surface 402 (e.g., surface of the skull 404) in a portion of the recipient's body (e.g., the head). The at least one implantable housing 310 is substantially parallel to the bone surface 402 (e.g., schematically illustrated in FIG. 2A by the dashed line 403a extending along the bone surface 402). For example, the at least one implantable housing 310 is compatible with (e.g., conforms to; follows) a curvature of the bone surface 402 in the first region 410 or the bone surface 402 can be altered (e.g., machined) to provide a bone surface 402 in the first region 410 with which the at least one implantable housing 310 is compatible.

The circuitry 312 of the at least one implantable housing 310 can comprise at least one internal energy reception coil 313 (e.g., a planar electrically conductive wire with multiple windings) and control circuitry 314 (e.g., at least one battery and/or capacitor and at least one microprocessor). The at least one internal energy reception coil 313 can be configured to wirelessly and transcutaneously receive power from an external source (e.g., the energy transmission coil 462 of the at least one external device 450). The control circuitry 314 can be configured to receive, store, and/or use the power from the at least one internal energy reception coil 313 and to control the one or more active elements of the apparatus 300.

The at least one implantable housing 310 can further comprise at least one internal magnetic (e.g., ferromagnetic; ferrimagnetic; permanent magnet; diamagnetic magnet) material 315 (e.g., disk; plate) configured to establish a magnetic attraction between the external magnetic material 464 of the at least one external device 450 with sufficient strength to hold the at least one external device 450 against an outer surface of the skin 406. For example, as shown in FIG. 2A, the at least one internal magnetic material 315 can be positioned within a region at least partially bounded by the at least one internal energy reception coil 313. As shown in FIG. 2A, the at least one internal energy reception coil 313 and the at least one internal magnetic material 315 can be within a first housing portion 316a of the at least one implantable housing 310 and the control circuitry 314 can be within a second housing portion 316b of the at least one implantable housing 310, the second housing portion 316b separate from but connected to the first housing portion 316a. Alternatively, the at least one implantable housing 310 can comprise a single housing portion configured to contain the at least one internal energy reception coil 313, the at least one internal magnetic material 315, and the control circuitry 314. The one or more active elements (not shown in FIGS. 2A and 2B) of the apparatus 300 can be separate from the at least one implantable housing 310 containing the circuitry 312 but can be within another implantable housing operationally coupled to one or more components within the at least one implantable housing 310 (e.g., the one or more active elements can be in electrical communication with the circuitry 312 via at least one elongate electrical lead 317).

In certain implementations, the at least one electrical conduit 320 comprises at least one electrically conductive wire having a first end portion in electrical communication with the circuitry 312 (e.g., the control circuitry 314 within the second housing portion 316b) and a second end portion in electrical communication with the at least one MI antenna 330. A length of the at least one electrical conduit 320 can be in a range of 5 millimeters to 40 millimeters. The at least one electrical conduit 320 can extend from a first region 410 of the recipient's body containing the at least one implantable housing 310 to a second region 420 of the recipient's body containing the at least one MI antenna 330. The at least one electrical conduit 320 can comprise multiple electrical conduits (e.g., two electrical conduits for each MI antenna 330). While FIG. 2A schematically illustrates the bone surface 402 in the first region 410 and the second region 420 being substantially flat and substantially parallel to one another, in certain other implementations, the bone surface 402 in the first region 410 and the bone surface 402 in the second region 420 are substantially non-parallel to one another and/or one or both are curved.

In certain implementations, the at least one electrical conduit 320 is malleable (e.g., bendable) such that a position and/or an orientation of the at least one MI antenna 330 relative to the at least one implantable housing 310 can be controllably adjusted during an implantation process. For example, as shown in FIG. 2A. the at least one electrical conduit 320 can comprise one or more bends such that the at least one implantable housing 310 is on a bone surface 402 within the first region 410 of the recipient's body (e.g., on a temporal bone surface of the skull 404) and the at least one MI antenna 330 is within the second region 420 (e.g., within a mastoid cavity beneath the temporal bone surface 402) with the antenna axis 332 of the at least one MI antenna 330 substantially non-parallel and substantially non-orthogonal to the bone surface 402.

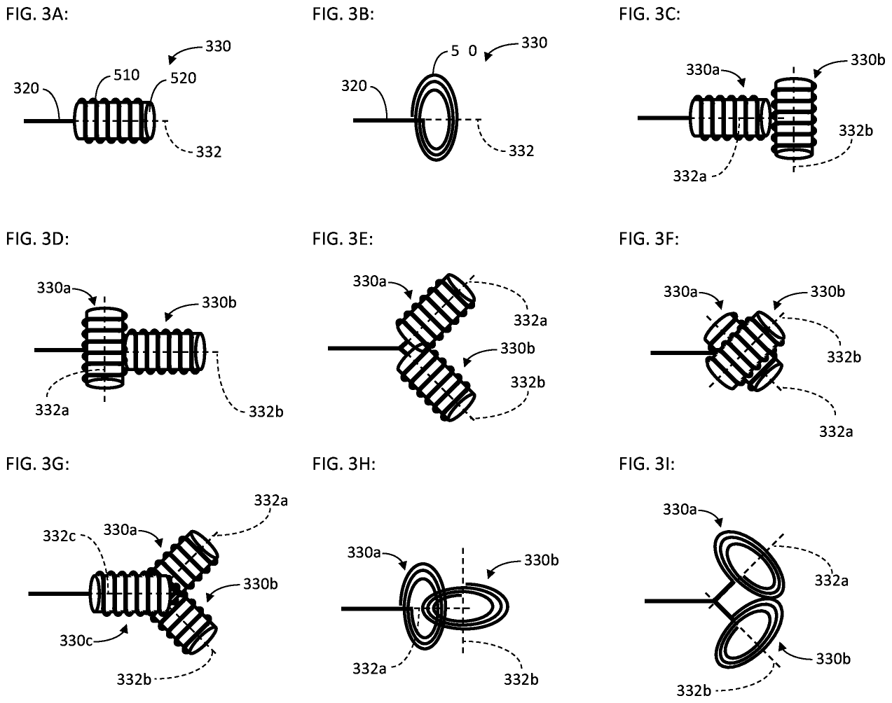

FIGS. 3A-3I schematically illustrate various examples of the at least one MI antenna 330 in accordance with certain implementations described herein. Although not shown in FIGS. 3A-3I, in certain implementations in which the at least one MI antenna 330 comprises non-biocompatible materials, the at least one MI antenna 330 comprises a biocompatible housing (e.g., silicone; polymer; ceramic; glass) configured to hermetically seal the non-biocompatible materials from an environment surrounding the housing. In addition, the housing can be substantially transparent to the electromagnetic or magnetic fields generated or received by the at least one MI antenna 330 such that the housing does not substantially interfere with the data transmission to and/or from the apparatus 300. In certain implementations, the at least one MI antenna 330 is configured to be implanted (e.g., affixed) within the second region 420. For example, the housing of the at least one MI antenna 330 can be configured to be affixed to a position within the recipient's body (e.g., using at least one biocompatible anchor, screw, or adhesive).

In certain implementations, the at least one MI antenna 330 is sufficiently small to fit within the volume of the second region 420 in which the at least one MI antenna 330 is to be implanted. For example, to be implanted within a mastoid cavity, as schematically illustrated in FIG. 2A, the dimensions of the at least one MI antenna 330 can be in a range of 4 millimeters to 15 millimeters (e.g., in a range of 5 millimeters to 7 millimeters). Once implanted within the mastoid cavity, the at least one MI antenna 330 is configured to be spaced from, but in inductive communication with, at least one external MI antenna 472 of the external device 450. For example, the at least one MI antenna 330 can have a center-to-center distance from the at least one external MI antenna 472 in a range of 2 millimeters to 15 millimeters.

In certain implementations, the at least one MI antenna 330 comprises a single substantially cylindrical wire coil 510 wound around and extending along the antenna axis 332. For example, as schematically illustrated by FIG. 3A, the at least one MI antenna 330 comprises a substantially cylindrical ferrous core 520 extending along the antenna axis 332 and the wire coil 510 is wound around the core 520 and is electrically insulated from the core 520. An electrical current flowing through the wire coil 510 can generate a magnetic dipole moment that is substantially coincident with the antenna axis 332. The wire coil 510 (e.g., an electrically insulated single-strand or multi-strand platinum or gold wire) can have a plurality of coil windings (e.g., 2, 3, 4, 5, 6, or more) around the antenna axis 332. While FIG. 3A schematically illustrates the substantially cylindrical wire coil 510 and core 520 having substantially circular cross-sections in a plane perpendicular to the antenna axis 332, in certain other implementations, the cross-sections of the substantially cylindrical wire coil 510 and core 520 have other shapes (e.g., oval; rectangular; triangular; polygonal; irregular). In certain implementations, the wire coil 510 and core 520 have a cross-sectional width (e.g., diameter; in a plane substantially perpendicular to the antenna axis 332) in a range of 1 millimeter to 5 millimeters (e.g., 2 millimeters to 4 millimeters), and an inductance in a range of 200 nH to 800 nH (e.g., about 500 nH).

In certain other implementations, the at least one MI antenna 330 comprises a single substantially planar wire spiral 530 wound around and substantially orthogonal to the antenna axis 332. An electrical current flowing through the wire spiral 530 can generate a magnetic dipole moment that is substantially coincident with the antenna axis 332. The wire spiral 530 (e.g., an electrically insulated single-strand or multi-strand platinum or gold wire) can have a plurality of coil windings (e.g., 2, 3, 4, 5, 6, or more) around the antenna axis 332. While FIG. 3B schematically illustrates the substantially planar wire spiral 530 having a substantially circular shape in a plane perpendicular to the antenna axis 332, in certain other implementations, the substantially planar wire spiral 530 have other shapes (e.g., oval; rectangular; triangular; polygonal; irregular).

In certain implementations, the MI antenna 330 of FIG. 3A comprising the single substantially cylindrical wire coil 510 or the MI antenna 330 of FIG. 3B comprising the single substantially planar wire spiral 530 is configured to be affixed within the region 420 such that the MI antenna 330 substantially avoids being in a “dead zone” of the at least one external MI antenna 472 of the external device 470. Such dead zones are locations and/or orientations of the MI antenna 330 in which the flux linkage with the at least one external MI antenna 472 is substantially zero (e.g., the coupling factor and/or the mutual inductance between the MI antenna 330 and the at least one external MI antenna 472 is substantially zero). Such dead zones can occur at locations and orientations of the MI antenna 330 at which the ingoing magnetic flux and the outgoing magnetic flux from the at least one external MI antenna 472 substantially cancel one another. For example, such dead zones can exist when the MI antenna 330 is positioned at various locations and the magnetic dipole moments of the MI antenna 330 and the at least one external MI antenna 472 are substantially perpendicular to one another or when the MI antenna 330 is positioned at various other locations and the magnetic dipole moments of the MI antenna 330 and the at least one external MI antenna 472 are substantially parallel to one another.

As schematically illustrated by FIG. 2A, certain external devices 470 (e.g., a BTE sound processor) are configured to be worn by the recipient with a magnetic dipole moment 474 of the at least one external MI antenna 472 oriented to be substantially parallel to the bone surface 402 beneath the external device 470. The MI antenna 330 of certain such implementations is configured to be affixed within the mastoid cavity such that the antenna axis 332 (e.g., the magnetic dipole moment of the at least one MI antenna 330) is substantially non-parallel and substantially non-orthogonal to the bone surface 402 (e.g., in a range of greater than or equal to 10 degrees relative to the bone surface 402). In certain such implementations, as shown in FIG. 2B, the antenna axis 332 and the at least one electrical conduit 320 can be substantially planar with one another, with the plane substantially perpendicular to an end portion of the second housing portion 316b from which the at least one electrical conduit 320 extends from the second housing portion 316b. In addition, the location of the MI antenna 330 within the mastoid cavity can be selected such that the MI antenna 330 is expected to substantially avoid dead zones of the at least one external MI antenna 472 for expected positions and orientations of the external device 470 while being worn by the recipient.

For example, optimal locations and/or orientations of the MI antenna 330 can be calculated prior to the implantation process, based on analysis of the recipient's anatomy to determine the most likely position and orientation of the at least one external MI antenna 472, and these optimal locations and/or orientations can be communicated to the practitioner performing the implantation (e.g., in an implantation guide or instructions) by reference to anatomical landmarks (e.g., the external auditory canal, ossicles, round window). For another example, an optimal location and/or orientation of the MI antenna 330 can be determined by the practitioner during the implantation process by using a feedback system in which an external MI antenna 472 is placed in the most likely position and orientation and is used to generate a time-varying magnetic field and the MI antenna 330 is moved around within the second region 420 to find a location and/or orientation of the MI antenna 330 which yields a optimal (or at least acceptable) MI coupling to the external MI antenna 472.

In certain implementations, the at least one MI antenna 330 comprises multiple MI antennas 330 with the antenna axes 332 of the MI antennas 330 substantially non-parallel to one another. For example, the at least one MI antenna 330 schematically illustrated by FIGS. 3C-3I each comprise a first MI antenna 330a comprising a first electrically conductive wire extending around a first antenna axis 332a and a second MI antenna 330b comprising a second electrically conductive wire extending around a second antenna axis 332b, the second antenna axis 332b at an angle greater than or equal to 45 degrees relative to the first antenna axis 332a (e.g., the second antenna axis 332b substantially orthogonal to the first antenna axis 332a). The first and second MI antennas 330a, b can be affixed to one another (e.g., both within a common housing).

In FIGS. 3C-3F, the first wire of the first MI antenna 330a comprises a substantially cylindrical first wire coil wound around and extending along the first antenna axis 332a and the second wire of the second MI antenna 330b comprises a substantially cylindrical second wire coil wound around and extending along the second antenna axis 332b. In FIGS. 3H and 3I, the first wire of the first MI antenna 330a comprises a substantially planar first wire spiral wound around and substantially orthogonal to the first antenna axis 332a and the second wire of the second MI antenna 330b comprises a substantially planar second wire spiral wound around and substantially orthogonal to the second antenna axis 332b. While not shown in FIGS. 3A-3I, in certain implementations, one or more MI antennas with substantially cylindrical wire coils can be used with one or more other MI antennas with substantially planar wire spirals.

As schematically illustrated by FIG. 3G, the at least one MI antenna 330 can further comprise a third MI antenna 330c comprising a third electrically conductive wire extending around a third antenna axis 332c. The third antenna axis 332c can be at an angle greater than or equal to 45 degrees relative to the first antenna axis 332a (e.g., substantially orthogonal to the first antenna axis 332a) and at an angle greater than or equal to 45 degrees relative to the second antenna axis 332b (e.g., substantially orthogonal to the second antenna axis 332b). For example, the third antenna axis 332c can be substantially orthogonal to the plane defined by the first antenna axis 332a and the second antenna axis 332b.

In certain implementations, the at least one MI antenna 330 is configured to be in operative communication with the at least one external MI antenna 472 of an external device 470 (e.g., BTE sound processor). The at least one MI antenna 330 can bound an antenna region through which magnetic flux from the at least one external MI antenna 472 extends. For example, the wire coil 510 of FIG. 3A bounds a substantially cylindrical volume and the wire coil 510 can be positioned within the recipient's body such that at least a portion of the magnetic flux extends through the volume in a direction substantially parallel to an axis of the substantially cylindrical volume (e.g., the antenna axis 332; a longitudinal axis). For another example, the wire spiral 530 of FIG. 3B bounds a substantially planar area and the wire spiral 530 can be positioned within the recipient's body such that at least a portion of the magnetic flux extends through the area in a direction substantially non-parallel to an axis of the area (e.g., the antenna axis 332; a symmetry axis).

In certain implementations in which the at least one MI antenna 330 comprises multiple MI antennas 330, by virtue of at least two of the MI antennas 330 having antenna axes 332 that are substantially perpendicular to one another, at least one of the multiple MI antennas 330 bounds an antenna region through which magnetic flux from the at least one external MI antenna 472 extends. For example, the substantially cylindrical volume bounded by at least one of the wire coils 510 of FIGS. 3C-3G has the magnetic flux from the at least one external MI antenna 472 extending therethrough in a direction substantially parallel to the axis of the substantially cylindrical volume (e.g., antenna axis 332). For another example, the substantially planar area bounded by at least one of the wire coils 530 of FIGS. 3H-3I has the magnetic flux from the at least one external MI antenna 472 extending therethrough in a direction substantially non-parallel to an axis of the area (e.g., the antenna axis 332).

In certain implementations in which the at least one MI antenna 330 comprises multiple MI antennas 330, the circuitry 312 of the apparatus 300 comprises antenna selection circuitry in operative communication with the multiple MI antennas 330. The antenna selection circuitry is configured to detect the amplitude of the electric voltage and/or electric current induced within each of the multiple MI antennas 330 by the magnetic flux generated by the at least one external MI antenna 472, to determine which of the multiple MI antennas 330 has the largest amplitude of the induced electric voltage and/or electric current, and to select the induced electric voltage and/or electric current having the largest amplitude for use by the apparatus 300.

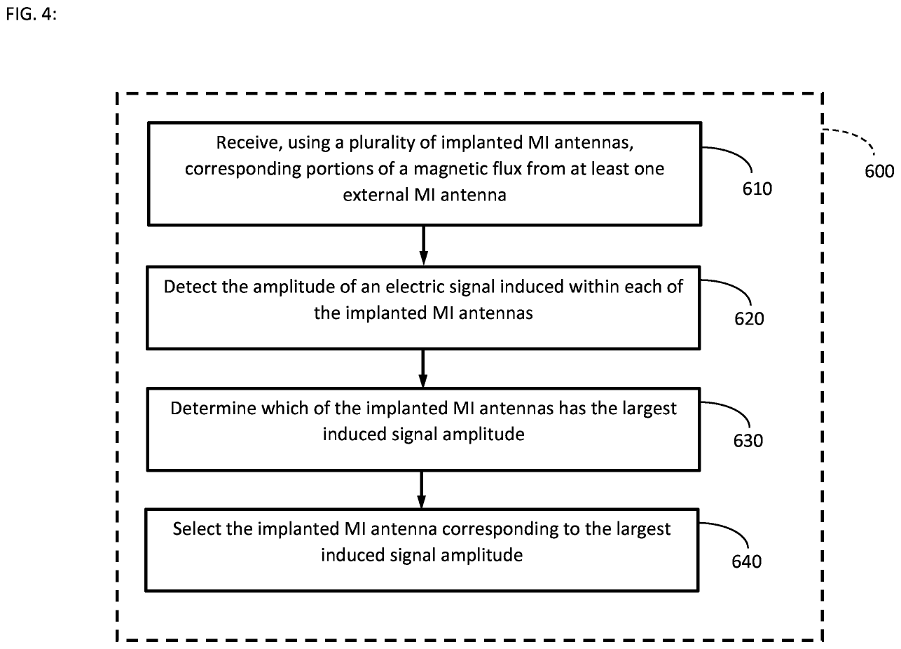

FIG. 4 is a flow diagram of an example method 600 in accordance with certain implementations described herein. While the method 600 is described by referring to some of the structures of the example apparatus 300 of FIGS. 2A-2B and 3A-3I, other apparatus and systems with other configurations of components can also be used to perform the method 600 in accordance with certain implementations described herein.

In an operational block 610, the method 600 comprises receiving, using a plurality of implanted MI antennas 330, corresponding portions of a magnetic flux from at least one external MI antenna 472. At least two of the implanted MI antennas 330 can have antenna axes 332 that are substantially non-parallel to one another (e.g., substantially orthogonal to one another). The implanted MI antennas 330 can be within a recipient's body, and the at least one external MI antenna 472 can be part of an external device outside the recipient's body. The corresponding portion of the magnetic flux received by an implanted MI antenna 330 can be the portion of the magnetic flux extending through an antenna region of the implanted MI antenna 330 in a direction along an axis of the implanted MI antenna 330 (e.g., having a non-zero component that is parallel to the antenna axis 332).

In an operational block 620, the method 600 further comprises detecting the amplitude of an electric signal (e.g., voltage and/or current) induced within each of the implanted MI antennas 330 by the corresponding received portion of the magnetic flux. At least one of the implanted MI antennas 330 has an induced signal with a non-zero amplitude. Each of the implanted MI antennas 330 receiving a portion of the magnetic flux extending at least partially along the axis of the implanted MI antenna 330 has an induced signal with a non-zero amplitude.

In an operational block 630, the method 600 further comprises determining which of the implanted MI antennas 330 has the largest induced signal amplitude. For example, a comparison of each of the induced signal amplitudes from each of the implanted MI antennas 330 can identify the largest induced signal amplitude and can then identify which of the implanted MI antennas 330 corresponds to this largest induced signal amplitude.

In an operational block 640, the method 600 further comprises selecting the implanted MI antenna 330 corresponding to the largest induced signal amplitude. The selected implanted MI antenna 330 can then be used by the apparatus 300 as a source of information (e.g., data and/or commands) and/or power from the external device for operation of the apparatus 300. For example, for an implanted portion of an auditory prosthesis system, the selected implanted MI antenna 330 can be used as a source of data and/or commands from an external portion of the auditory prosthesis system.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of various devices, various implementations described herein can be incorporated in a variety of other suitable devices, methods, and contexts. More generally, as can be appreciated, certain implementations described herein can be used in a variety of implantable medical device contexts that can benefit from certain attributes described herein.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein but should be defined only in accordance with the claims and their equivalents.

1. An apparatus comprising:
at least one implantable housing containing circuitry and configured to be implanted on and substantially parallel to a bone surface within a recipient;
at least one electrical conduit in electrical communication with the circuitry and extending from the at least one implantable housing to a region within the recipient; and
at least one magnetic induction antenna in electrical communication with the at least one electrical conduit and spaced from the at least one implantable housing, the at least one magnetic induction antenna extending around an antenna axis, the at least one magnetic induction antenna configured to be affixed within the region with the antenna axis substantially non-parallel and substantially non-orthogonal to the bone surface.

2. The apparatus of claim 1, wherein the at least one magnetic induction antenna comprises a single substantially cylindrical wire coil wound around and extending along the antenna axis.

3. The apparatus of claim 1, wherein the at least one magnetic induction antenna comprises a single substantially planar wire spiral wound around and substantially orthogonal to the antenna axis.

4. The apparatus of claim 2, wherein the antenna axis is at an angle greater than or equal to 10 degrees relative to the bone surface.

5. The apparatus of claim 1, wherein the at least one magnetic induction antenna comprises a first magnetic induction antenna comprising a first electrically conductive wire extending around a first antenna axis and a second magnetic induction antenna comprising a second electrically conductive wire extending around a second antenna axis, the second antenna axis at an angle greater than or equal to 45 degrees relative to the first antenna axis.

6. The apparatus of claim 5, wherein the first wire comprises a substantially cylindrical first wire coil wound around and extending along the first antenna axis or a substantially planar first wire spiral wound around and substantially orthogonal to the first antenna axis.

7. The apparatus of claim 6, wherein the second wire comprises a substantially cylindrical second wire coil wound around and extending along the second antenna axis or a substantially planar second wire spiral wound around and substantially orthogonal to the second antenna axis.

8. The apparatus of claim 5, wherein the second antenna axis is substantially orthogonal to the first antenna axis.

9. The apparatus of claim 5, wherein the at least one magnetic induction antenna further comprises a third magnetic induction antenna comprising a third electrically conductive wire extending around a third antenna axis, the third antenna axis at an angle greater than or equal to 45 degrees relative to the first antenna axis and at an angle greater than or equal to 45 degrees relative to the second antenna axis.

10. The apparatus of claim 1, wherein the at least one magnetic induction antenna is configured to be in operative communication with at least one external magnetic induction antenna of a device, the at least one magnetic induction antenna bounding an antenna region through which magnetic flux from the at least one external magnetic induction antenna extends.

11. The apparatus of claim 10, wherein the at least one magnetic induction antenna comprises a wire coil and the antenna region comprises a substantially cylindrical volume, at least a portion of the magnetic flux extending through the volume in a direction substantially parallel to an axis of the substantially cylindrical volume.

12. The apparatus of claim 10, wherein the at least one magnetic induction antenna comprises a wire spiral and the antenna region comprises a substantially planar area, at least a portion of the magnetic flux extending through the area in a direction substantially non-parallel to an axis of the area.

13. The apparatus of claim 10, wherein the bone surface comprises a skull surface, the region comprises a mastoid cavity, and the device is on and/or behind a pinna of the recipient, wherein the apparatus is an implanted portion of an acoustic prosthesis system and the device is an external portion of the acoustic prosthesis system.

14. A method comprising:
receiving, using a plurality of implanted magnetic induction antennas, corresponding portions of a magnetic flux from at least one external magnetic induction antenna;
detecting the amplitude of an electric signal induced within each of the implanted magnetic induction antennas by the corresponding received portion of the magnetic flux;
determining which of the implanted magnetic induction antennas has the largest induced signal amplitude; and
selecting the implanted magnetic induction antenna corresponding to the largest induced signal amplitude.

15. The method of claim 14, wherein at least two of the implanted magnetic induction antennas have antenna axes that are substantially orthogonal to one another.

16. The method of claim 14, wherein the corresponding portion of the magnetic flux received by an implanted magnetic induction antenna is the portion of the magnetic flux extending through an antenna region of the implanted magnetic induction antenna having a non-zero component that is substantially parallel to an antenna axis of the implanted magnetic induction antenna.

17. The method of claim 14, wherein the induced signal amplitude comprises an induced electric voltage amplitude and/or an induced electric current amplitude.

18. The method of claim 14, wherein at least one of the implanted magnetic induction antennas has an induced signal with a non-zero amplitude.

19. The method of claim 14, wherein said determining comprises identifying the largest induced signal amplitude and identifying which of the implanted magnetic induction antennas corresponds to the largest induced signal amplitude.

20. The method of claim 14, wherein the implanted magnetic induction antennas are within a recipient's body, and the at least one external magnetic induction antenna is part of an external device outside the recipient's body.

21. The method of claim 14, further comprising using the selected implanted magnetic induction antenna as a source of information for operation of the apparatus.

22. An apparatus comprising:
at least one external device configured to be worn by a recipient, the at least one external device comprising at least one external magnetic induction antenna configured to generate a magnetic flux; and
at least one implantable device comprising circuitry and at least a first magnetic induction antenna and a second magnetic induction antenna in electrical communication with the circuitry, the first and second magnetic induction antennas affixed to one another, the first magnetic induction antenna having a first antenna axis and the second magnetic induction antenna having a second antenna axis substantially non-parallel to the first antenna axis.

23. The apparatus of claim 22, wherein at least one of the first magnetic induction antenna and the second magnetic induction antenna comprises a substantially cylindrical wire coil wound around and extending along the antenna axis.

24. The apparatus of claim 22, wherein at least one of the first magnetic induction antenna and the second magnetic induction antenna comprises a substantially planar first wire spiral wound around and substantially orthogonal to the antenna axis.

25. The apparatus of claim 22, wherein the first antenna axis is substantially orthogonal to the second antenna axis.
